(19) United States
(12) Patent Application Publication
(10) Pub. No.: US 2023/0410991 A1
(43) Pub. Date: Dec. 21, 2023

(54) CRYOGENIC STORAGE TRANSPORTATION TRACKING SYSTEM

(71) Applicant: JANSSEN BIOTECH, INC., Horsham, PA (US)

(72) Inventors: Michael HUTCHINSON, King of Prussia, PA (US); Jason TANSKY, Collegeville, PA (US)

(21) Appl. No.: 18/250,695

(22) PCT Filed: Oct. 28, 2021
(86) PCT No.: PCT/IB2021/059986

§ 371 Date: Apr. 26, 2023

Related U.S. Application Data
(60) Provisional application No. 63/107,021, filed on Oct. 29, 2020.

(51) Int. Cl. G16H 40/20; G06K 19/07; A61J 1/18; A61J 1/16; A01N 1/02

(52) U.S. Cl. CPC G16H 40/20; G06K 19/07; A61J 2205/60; A61J 1/165; A01N 1/0257; A61J 1/18

(57) ABSTRACT

A system for wireless identification of pharmaceutical products stored in a storage device is disclosed. The system includes a plurality of racks adapted to hold a plurality of pharmaceutical products, wherein each rack comprises one or more apertures; a plurality of remotely readable tags each adapted to be affixed to one or more of the pharmaceutical products, and at least one antenna operatively connected to a reader configured to interrogate the remotely readable tags. The reader is operatively connected to a computerized inventory system that is configured to receive information about each remotely readable tag, and the reader detects a position of each remotely readable tag and associated pharmaceutical product on the plurality of racks.

### CROSS-REFERENCE TO RELATED APPLICATIONS

The present application claims priority to U.S. Provisional Application No. 63/107,021 filed on Oct. 29, 2020, the entire contents of which are incorporated herein by reference.

### BACKGROUND

In cell therapy and biologic based pharmaceuticals, it is a standard practice to store, both temporarily or long-term, in-process materials (material) or final drug products (product) at cryogenic temperatures in order to preserve the viability of the material and product. Proper and secure labelling and identification of containers carrying cell therapy and biologic based pharmaceuticals are pivotal for daily industrial operations including transportation.

Labelling and tracking such material and product is of vital importance: mixing up two different materials or products may have profound consequences. In addition, if the label for a material or product is lost, it may be difficult, costly and time-consuming, or perhaps even impossible, to then identify that material or product.

Biological products are often stored in a storage device, such as a vacuum flask, that contains coolant fluid, such as cryogenic fluid (e.g. liquid nitrogen). Certain types of biological products are required to be audited or inventoried in a manufacturing setting at regular intervals. However, when the materials or products are removed, albeit briefly, from cold storage in order for their identities to be checked, they will tend to warm up, which may reduce their storage lifetime. There is also a risk when removing the materials or products containers that they may be accidently dropped causing a loss of material.

Accordingly, there remains a need in the art for improved cryogenic storage transportation tracking systems.

### SUMMARY OF THE INVENTION

Generally, the present invention relates to a low or ultra-low temperature cryogenic storage device that includes inside of it one or more antennas, a plurality of racks, and a plurality of remotely readable tags. More specifically, embodiments of the present invention include a wireless identification system that includes an integrated radio frequency identification (RFID) or microelectromechanical system (MEMS) antennas inside the storage device. The antennas are installed inside a low temperature or ultra-low temperature storage device that may, upon being requested, sense and/or detect remotely readable tagged in-process materials (materials) or final drug products (products) encased inside secondary protective containers. The secondary protective containers disclosed herein may comprise cassettes or similar containers thereof.

In order for the antennas to properly read the plurality of remotely readable tags, the rack may have a plurality of apertures which may be of any shape or form. The rack may contain an aperture that has a circumference that is preferably at least a quarter of an inch. The apertures permit signals to transverse the rack from the antennas to the remotely readable tags.

The antennas may then transmit data from the plurality of remotely readable tags to a reader for material or product identification, organization, classification, databasing, tracking or locating. According to embodiments, a storage device contains an antenna inside of it where the antenna transmits data in real time for material or product identification, organization, tracking, and locating.

According to an embodiment, a system for wireless identification of pharmaceutical products stored in a storage device includes: a plurality of racks adapted to hold a plurality of pharmaceutical products, wherein each rack comprises one or more apertures; a plurality of remotely readable tags each adapted to be fixed to one or more of the pharmaceutical products; and at least one antenna operatively connected to a reader configured to interrogate the remotely readable tags, wherein the reader is operatively connected to a computerized inventory system that is configured to receive information about each remotely readable tag, and wherein the reader detects a position of each remotely readable tag and associated pharmaceutical product on the plurality of racks.

According to another embodiment, a system for wireless identification of pharmaceutical products stored in a storage device includes' a plurality of racks adapted to hold a plurality of pharmaceutical products; a plurality of remotely readable tags each adapted to be affixed to one or more of the plurality of pharmaceutical products; a plurality of secondary protective containers that comprise the plurality of pharmaceutical products; and at least one antenna operatively connected to a reader configured to interrogate the remotely readable tags, wherein the reader is operatively connected to a computerized inventory system that is configured to receive information about each remotely readable tag, and wherein the reader detects a position of each remotely readable tag and associated pharmaceutical product on the plurality of racks.

According to yet another embodiment, a system for wireless identification of pharmaceutical products stored in a storage device includes, a plurality of racks adapted to hold a plurality of pharmaceutical products; a plurality of remotely readable tags each adapted to be affixed to one or more of the plurality of pharmaceutical products; a plurality of secondary protective containers that contain the plurality of pharmaceutical products, wherein the secondary protective containers comprise a plurality of apertures; and at least one antenna operatively connected to a reader configured to interrogate the remotely readable tags, wherein the reader is operatively connected to a computerized inventory system that is configured to receive information about each remotely readable tag, and wherein the reader detects a position of each remotely readable tag and associated pharmaceutical product on the plurality of racks.

According to yet another embodiment, a system for wireless identification of pharmaceutical products stored in a storage device includes: a plurality of remotely readable tags affixed to a plurality of pharmaceutical products that are attached to a plurality of racks wherein the racks comprise a plurality of apertures; and a multi-array antenna operatively connected to a reader configured to interrogate the remotely readable tags; wherein the reader is operatively connected to a computerized inventory system that is configured to detect each remotely readable tag and wherein the reader detects a position of each remotely readable tag and associated pharmaceutical product on the plurality of racks.

According to yet another embodiment, a system for a wireless identification of a pharmaceutical products stored in a storage device includes: a plurality of remotely readable tags associated with a plurality of pharmaceutical products on a plurality of racks wherein each rack functions as an antenna and is operatively connected to a reader configured to interrogate the remotely readable tags; wherein the reader is operatively connected to a computerized inventory system that is configured to detect each remotely readable tag, and wherein the reader detects a position of each remotely readable tag and associated pharmaceutical product on each rack.

### BRIEF DESCRIPTION OF DRAWINGS

FIG. 1 is a partial cross-sectional view of a storage device suitable for use in a system for remote live auditing and/or inventorying in-process materials or final drug products.

FIG. 2 is a front elevation of a rack.

FIG. 3 is a perspective view of a cassette according to embodiments of the invention, adapted to receive a final drug product or in-process material therein.

FIG. 4 is an additional partial cross-sectional view of a storage device suitable for use in a system for remote live auditing and/or inventorying in-process materials or final drug products.

FIG. 5 is another partial cross-sectional view of a storage device suitable for use in a system for remote live auditing and/or inventorying in-process materials or final drug products.

### DEFINITIONS

When introducing elements of the present disclosure or the preferred embodiment(s) thereof, the articles “a”, “an”, and “the” when used herein are intended to mean that there are one or more of the elements.

The terms “comprising” “including” and “having” when used herein are intended to be inclusive and mean that there may be additional elements other than the listed elements.

The term “plurality” when used herein refers to one or more.

The terms “in-process material” and “final drug product” when used herein refer to blood bags, viruses, DNA libraries, tissue samples, commercial and/or non-commercial samples, specimens, products, containers and biologics and other cell therapies such as CAR-T cell therapy that are stored in a low temperature or ultra-low temperature storage device. The term “pharmaceutical products” as used herein is intended to capture “in-process material,” “final drug product” and other substances used in the pharmaceutical industry in the manufacture of therapeutic products.

### DETAILED DESCRIPTION

Embodiments of the present invention include a wireless identification system of in-process materials (materials) or final drug products (products) stored in a storage device. The wireless identification system includes an integrated radio frequency identification (RFID) or microelectromechanical systems (MEMS) antenna or any other communication technologies that may transmit information wirelessly between at least two components provided with antennas. Individual or multiple RFID or MEMS tagged materials or products may be sensed and/or detected simultaneously by the RFID or MEMS antenna. Alternatively, the RFID or MEMS tagged materials or products may be sensed and/or detected separately by the RFID or MEMS antenna.

An RFID or MEMS tag in connection with the present invention may be programmed with information pertaining to the materials or products, including, but not limited to, content and characteristics of the storage device and materials or products, categories and subcategories that materials or products belong to, location of the storage device, position and coordinates of the materials or products in the storage device, destination of the materials or products, modification and expiry date, date and time item was stored in, removed from, manipulated in or moved within the storage device, name of person storing material or product in, removing material or product from, manipulating material or product in or moving material or product within the storage device, tracking number, identification number, patient name or ID number, place, origin, chronology and history of item or item content or item content creation, treatments and modifications that an item content or item content source or item content host were subjected to, contact coordinates, references and information on owner, distributor or supplier, description of content, instructions, name of mutation, type of mutation, category of mutation, name of disease, type of disease, category of disease, tumor name, any pathological condition, name of species, name of organism, name of organ, name of body part, name of tissue, and name of cell.

Installation of an RFID or MEMS antenna or any similar communication technology thereof inside of a storage device allows automatic registration of a tag stored in, entering in, exiting from or moving within a storage device. Installation of such an antenna inside the storage device at low temperature or ultra-low temperatures and/or in and/or on its various internal locations, components, accessories and/or compartments allows an even higher level of control. For example, an antenna may be installed in an individual or entire rack, shelf, drawer, compartment or a section of a freezer, cryogenic freezer, refrigerator etc., and which tracks a material or product inside the storage device when the material or product is moved from one location to another.

Using simultaneous readings of multiple RFID or MEMS tags with a RFID or MEMS antenna, respectively, has many benefits for use inside low-temperature and ultra-low temperature storage devices. For example, in many conventional cases identification is done by a handheld RFID reading device whereby a person has to physically lift the material or product out of a liquid nitrogen tank by one hand and hold the reader in the other hand. In most cases some spilling of liquid nitrogen happens which makes the procedure very inconvenient and even hazardous. As is the case with embodiments of the present invention, when the antenna is installed inside the storage device, the tag can be read automatically without ever opening or entering the storage device. It is also inconvenient and in some cases difficult and not practical to carry a reader every time a material or product has to be added to, removed from or moved within the storage device and/or needs to be identified. It might cause delays in the process of identification and in some cases create hazardous situations such as getting severe burns by liquid nitrogen or by ultra-low temperatures for the person who is trying to hold a rack in one hand and identify a material or product with a reader in the other hand. Additionally, there is concern around potential loss of material or product due to mishandling and dropping of the material or product.

The present invention is not intended to be limited to any particular type of retrievable and readable RFID or MEMS tag, antennae or reading device. The present invention covers all components of an RFID or MEM system that employ the concept(s) of RFID or MEMS technology and its development in the future.

Additionally, due to a wide variety of materials or products that may be stored in commercial and non-commercial settings, the present invention is not intended to be limited to a specific material or product mentioned and is intended to cover any material or product that may be stored in a low temperature or ultra-low temperature storage device.

Embodiments of the present invention can provide functionality beyond keeping simple inventory. According to embodiments, the present invention facilitates rapid identification, location and subsequent retrieval of material or product in a low temperature or ultra-low temperature storage device in a dynamic environment, for example, in an industrial manufacturing environment. With embodiments of the present invention, such information may be provided in real-time. Real-time information on important materials, products and/or materials are essential for efficient functioning in an industrial setting. The storage device may be autonomous or integrated in and/or associated with a computer setting which can detect and/or keep track of any tagged secondary protective container such as, for example, tracking during transportation and storage.

According to embodiments, the present invention can help to eliminate human error and make the identification process more reliable and accurate. The information obtained from the remotely readable tag is stored in the memory of a computer or other device that keeps an accurate log on the storage device number and location, material or product number and location inside the storage device, date, time and even the person who manipulated the item (for example, in instances where access to the storage device is limited by security access cards). Subsequently, by entering the ID or any other parameter of the material, or product it is easily identified, located and subsequently retrieved, as compared to the very laborious and time consuming exercise of manually searching for an item among hundreds or thousands of items stored in a single storage device or even worse, within multiple storage devices.

In addition, embodiments of the present invention save time due to eliminating the need for locating a reader and eliminating the time required to go back to take it when the person forgot to bring it with him or her. In addition, it significantly reduces the time required for scanning of multiple RFID or MEMS tagged materials or products. Embodiments of the present invention facilitate the handing and manipulation of multiple materials or products. Embodiments of the present invention also increase the reliability and accuracy of material or product identification since it will not depend on each person accurately detecting every material or product (for example, in some cases a person might forget to scan a material or product), but rather it is done automatically.

Furthermore, an information management system for identifying, tracking or locating materials or products stored in any number of different storage devices, which may be centrally located or located at different locations, may be generated by linking the storage devices in a network. The linking may be via wire or wireless connection. Regardless, linking the network allows for combining the variable information of each material or product in each storage device into a single list, for centralized access or searching thereof.

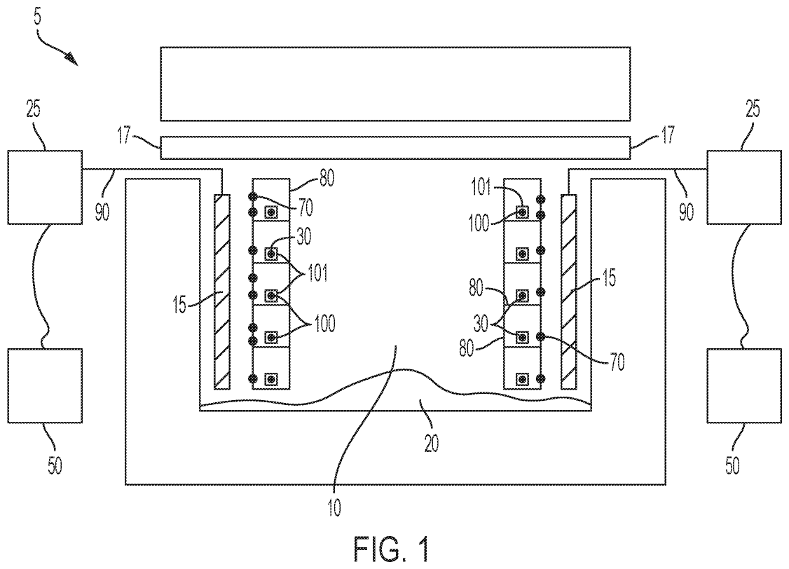

FIG. 1 illustrates an example embodiment of a wireless identification system 5 that allows the remote live auditing and/or inventorying of materials or products while contained in a storage device 10 filled with coolant fluid 20. Examples of a storage device disclosed herein may include a vacuum flask such as a cryogenic storage Dewar. FIG. 1 is a partial cross-sectional view of a storage device 10 and shows the components of one embodiment of the system present in the current disclosure.

In the particular example shown in FIG. 1, the storage device 10 is a cryogenic storage Dewar. The storage device 10 may thermally insulate the contents therein from the outside environment, which assists the coolant fluid 20 in maintaining the materials or products 100 in a cooled state.

The contents of the storage device 10 may be maintained at a temperature that is significantly below room temperature for long periods of time. To further assist in maintaining the low temperature within the storage device 10, the coolant fluid 20 within the storage device 10 may be replaced periodically. For instance, the coolant fluid 20 may evaporate over time and further coolant fluid 20 in a liquid state and at lower temperature may be added to replace the evaporated fluid.

The coolant fluid may be a cryogenic coolant fluid such as, for example, liquid nitrogen. Liquid nitrogen is commonly used because it is in a liquid state at extremely low temperature, having a freezing point of 63K. Further, liquid nitrogen's very low boiling point of 77K means that it may be kept in the flask in an essentially constantly slow-boiling state, which results in it maintaining a roughly constant temperature around its boiling point.

As may be seen from FIG. 1, the system may include two antennas 15. The system may also include one or multiple antennas 15. The antennas 15 are configured so that they may be placed on the far right and far left sides inside the storage device 10. Alternatively, the antennas 15 may be placed anywhere inside of the storage device 10.

The antennas 15 disclosed in FIG. 1 emit radio signals to activate the RFID or MEMS plurality of remotely readable tag(s) 30 on the materials or products 100 and read and write data to it. Antennas 15 are the conduits between the tag(s) 30 and the reader 25. The reader 25 controls the system's data acquisition and communication. The antennas 15 emit radio waves in ranges of anywhere from about one inch to 100 feet or more, depending upon its power output and the radio frequency used. When an RFID or MEMS tag passes through the electromagnetic zone, it detects the reader's 25 activation signal. The reader 25 decodes the data encoded in the integrated circuit (chip) of tag 30 and the data may then be passed wirelessly or wired for processing though an inventory management system 50.

Additionally, according to the setup in FIG. 1, in order for the antennas 15 to communicate with the plurality of remotely readable tags 30, a plurality of apertures 70 are made in or on a rack 80 facing the antennas 15. The apertures 70 may have a circumference of at least about a quarter of an inch to obtain an optimal signal. The apertures 70 may be of any shape or form. According to embodiments, the apertures 70 placed on the rack 80 should allow direct access to the antennas 15 and the plurality of remotely readable tags 30 so as to obtain a strong and consistent signal. The number of apertures 70 on the rack may be one, two, five, ten, twenty or more. The number of apertures 70 to be placed on the rack 80 is dependent on strength and signal consistency achieved between the antennas 15 and the remotely readable tag(s) 30.

In addition to apertures 70 made on the rack 80, apertures 70 may be made in or on a secondary protective container 101 so as to further achieve a strong and consistent signal between the antennas 15 and the remotely readable tag(s) 30. The remotely readable tags 30 are attached or placed on or next to the materials or products 100. Both the remotely readable tags 30 and materials or products 100 are located inside the secondary protective container 101.

Further, in view of FIG. 1, the antennas 15 and the readers 25 may be connected to each other through an electrical cord, flat wire 90 or cable. One or two storage door gaskets 17 may be deployed above, below or both above and below the flat wire 90 as depicted in FIG. 1. The storage door gaskets 17 used herein are any standard mechanical seal known in the art. The storage door gasket(s) 17 may be compressed between a storage door and a storage vessel of the storage device 10 to seal the interior of the storage device 10.

Furthermore, the antennas 15 automatically register and keep track of any material or product 100 which is stored in a storage device 10 such as in a cryogenic tank. The antennas 15 may be built into, integrated in or installed on any internal permanent or readable part of the tank which will allow automatic identification of a material or product stored or put into, removed from and/or moved within the storage device 10. The movement of secondary protective containers 101 with an RFID or MEMS tag(s) 30 are sensed by the antenna(s) 15 which transmits the signal to the readers 25. The readers 25 may be connected to an inventory management system 50 which keeps track of all materials or products 100 and allows rapid identification, tracking and/or location of the material or product 100.

Additionally, in view of FIG. 1, the use of one antenna 15 in a storage device 10 is possible in a wireless identification system setup as disclosed herein. Further, more than two antennas 15 in a storage device 10 is possible in a wireless identification system setup as disclosed herein.

Further, in view of FIG. 1, part of the coolant fluid 20 may be in a gaseous form because of evaporation. As the resulting gas will also typically be at a low temperature, it may not be necessary for the plurality of rack(s) 80 and plurality of secondary protective containers 101 to be submerged within the liquid part of the coolant fluid 20. Indeed, it is common practice for rack(s) 80 and secondary protective containers(s) 101, within a storage device 10, to be kept in the gas part of the coolant fluid 20.

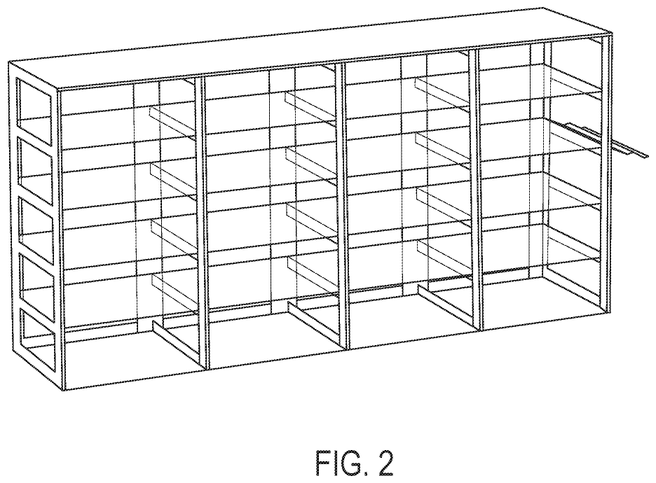

The rack 80 used herein may be any standard rack used in cryogenic conditions known in the industry. The rack 80 may have closed or open side panels. FIG. 2 depicts a rack with open side panels. Each rack 80 may be slotted so that the secondary protective container 101 may fit directly into each rack. Alternatively, each rack 80 may have a flat surface to hold the secondary protective container 101 as depicted in FIG. 2. Or each rack 80 may be designed to include a hook so as to hang the secondary protective container 101. Alternative structures known in the art to support the protective secondary container 101 or material or product 100 on the rack are also contemplated.

According to embodiments having a rack 80 as depicted in FIG. 2, there may be no need for apertures 70 on the rack 80. Apertures may alternatively be made on the secondary protective container 101 so as to optimize communication between the remotely readable tag(s) 30 and the antenna(s) 15. The number of apertures made on the secondary protective container 101 may be one, two, five, ten or more. The number of apertures is dependent upon the signal consistency and strength. The size of the aperture(s) 70 on the secondary protective container 101 is at least one millimeter. The material that the secondary protective container 101, rack(s) 80, and materials and products 100 are made from are any known material that may withstand cryogenic conditions.

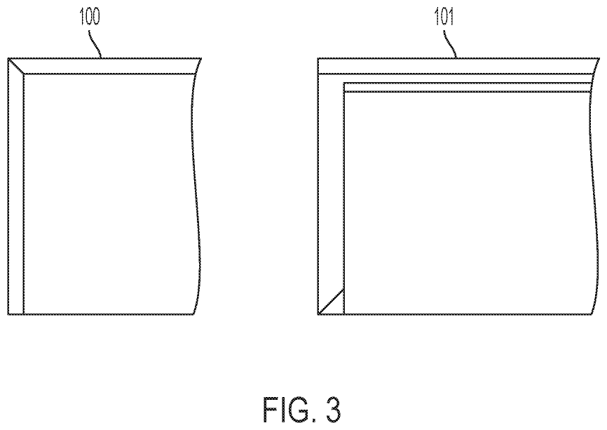

FIG. 3 depicts a cassette container which is an example of a secondary protective container 101. The cassette container contains the material or product 100.

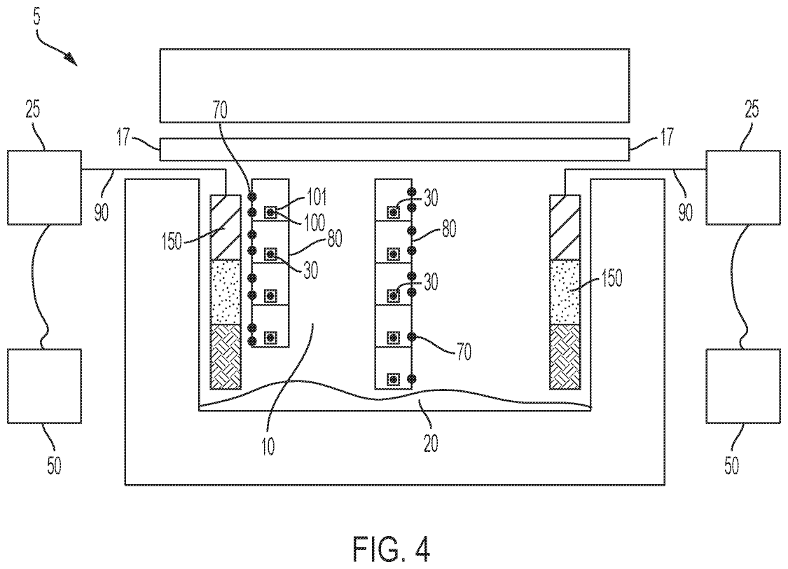

FIG. 4 additionally illustrates an example of an embodiment of a wireless identification system 5 that allows the remote live auditing or inventorying of materials or products while contained in a storage device 10 filled with coolant fluid 20. FIG. 4 is a partial cross-sectional view of a storage device 10 and shows the components of another embodiment of the system present in the current disclosure.

The wireless identification system of FIG. 4 is the same as that of FIG. 1 except that FIG. 4 includes multi-array antennas 150 as opposed to two single stand-alone antenna as set forth in FIG. 1. As may be seen from FIG. 4, the multi-array antennas 150 are configured so that they may be placed on the far right and far left sides inside the storage device 10. Additionally, the antennas 150 may be placed anywhere inside of the storage device 10.

The antennas 150 disclosed in FIG. 4 emit radio signals to activate the RFID or MEMS plurality of remotely readable tag(s) 30. The multi-array antennas 150 are the conduits between the tag(s) 30 and the readers 25. The reader 25 controls the system's data acquisition and communication. The multi-array antennas 150 emit radio waves in ranges of anywhere from about one inch to 100 feet or more, depending upon its power output and the radio frequency used. When an RFID or MEMS tag passes through the electromagnetic zone, it detects the reader's 25 activation signal. The reader 25 decodes the data encoded in the integrated circuit (chip) of the tag 30 and the data may then be passed wirelessly or wired for processing though an inventory management system 50.

Additionally, according to the set up in FIG. 4, in order for the multi-array antennas 150 to communicate with the plurality of tags 30, a plurality of apertures 70 may be made in or on the side of the rack 80 facing the multi-array antennas 150. The apertures 70 may have a circumference of at least about a quarter of an inch. The apertures 70 may be in any shape or form. According to embodiments, the number of apertures 70 placed on the side of a rack 80 line up the antennas 15 and the plurality of tags 30 so as to obtain a strong and consistent signal. The rack 80 used herein may be any standard rack known in the industry that may be used in cryogenic conditions.

Further, in view of FIG. 4, the multi-array antennas 150 and the readers 25 may be connected to each other through a flat wire 90. As with the embodiment of FIG. 1, one or two storage door gaskets 17 may be deployed above, below or both above and below the flat wire 90 as depicted in FIG. 4. The storage door gaskets 17 used herein are any standard mechanical seal known in the art.

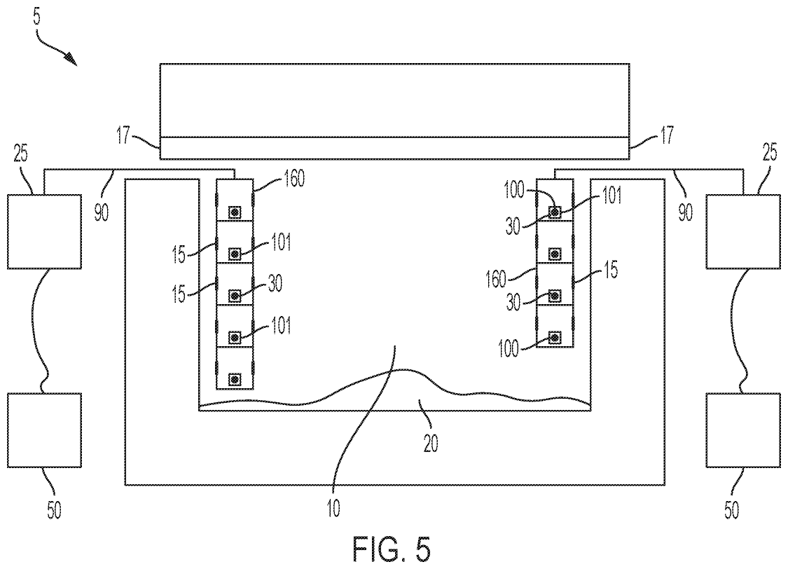

FIG. 5 also illustrates an example of an embodiment of a system that allows the remote live auditing or inventorying of materials or products while contained in a storage device 10 filled with coolant fluid 20. FIG. 5 is a partial cross-sectional view of a storage device 10 and shows the components of another embodiment of the system present in the current disclosure.

As may be seen from FIG. 5, unlike the wireless identification system 5 setups in FIGS. 1 and 4, the plurality of racks themselves serves as antennas inside the storage device 10. The plurality of rack antennas 160 may be placed anywhere inside of the storage device 10.

The rack antenna combination 160 disclosed in FIG. 5 emits radio signals to activate the RFID or MEMS plurality of remotely readable tag(s) 30. Rack antennas 160 are the conduits between the tag(s) 30 and the readers 25. The reader 25 controls the system's data acquisition and communication. The rack antennas 160 emits radio waves in ranges of anywhere from about one inch to 100 feet or more, depending upon its power output and the radio frequency used. When an RFID or MEMS tag passes through the electromagnetic zone, it detects the activation signal of the reader 25. The rack in the rack antenna combination 160 used herein may include any standard rack known in the industry that is used in cryogenic conditions with any standard antenna technology disclosed herein.

The reader 25 decodes the data encoded in integrated circuit (chip) of the tag 30 and the data may then be passed wirelessly or wired for processing though an inventory management system 50. Additionally, according to embodiments of FIG. 5, there may not be a need for a plurality of apertures 70 on the rack antennas 160 since the rack antennas directly interact with the plurality of remotely readable tags 30. However, apertures may alternatively be made on the secondary protective container 101 so as to optimize communication between the remotely readable tag(s) 30 and the antenna(s) 15. The number of apertures made on the secondary protective container 101 may be one, two, five, ten or more. The number of apertures is dependent upon the signal consistency and strength.

Further, in view of FIG. 5, the rack antennas 160 and the readers 25 may be connected to each other through a flat wire 90. As with the embodiment of FIG. 1, one or two storage door gaskets 17 may be deployed above, below or both above and below the flat wire 90 as depicted in FIG. 5. The storage door gaskets 17 used herein are any standard mechanical seal known in the art.

It should be understood that the present invention is by no means limited to the above-described embodiments. More generally, it should be appreciated that other examples and variations are contemplated within the scope of the appended claims. Furthermore, it should be noted that the foregoing description is intended to provide a number of non-limiting examples that assist the skilled reader's understanding of the present invention and that demonstrate how the present invention may be implemented.

What is claimed is:

1. A system for wireless identification of pharmaceutical products stored in a storage device comprising:
a plurality of racks adapted to hold a plurality of pharmaceutical products, wherein each rack comprises one or more apertures;
a plurality of remotely readable tags each adapted to be affixed to one or more of the pharmaceutical products, and
at least one antenna operatively connected to a reader configured to interrogate the remotely readable tags,
wherein the reader is operatively connected to a computerized inventory system that is configured to receive information about each remotely readable tag, and wherein the reader detects a position of each remotely readable tag and associated pharmaceutical product on the plurality of racks.

2. The wireless identification system of claim 1, wherein the plurality of apertures on the plurality of racks are in alignment with the antenna.

3. The wireless identification system of claim 1, wherein each of the plurality of pharmaceutical products is stored in a separate secondary protective container.

4. The wireless identification system of claim 3, wherein the secondary protective containers comprise a plurality of apertures.

5. The wireless identification system of claim 1, wherein the storage device includes one or more doors sealed by door gaskets, and the connection between the antenna and the reader comprises a flat cable that is positioned above, between, or under the door gaskets.

6. The wireless identification system of claim 1, wherein the remotely readable tags comprise of RFID or MEMS.

7. A system for wireless identification of pharmaceutical products stored in a storage device comprising:
a plurality of racks adapted to hold a plurality of pharmaceutical products;
a plurality of remotely readable tags each adapted to be affixed to one or more of the plurality of pharmaceutical products,
a plurality of secondary protective containers that comprise the plurality of pharmaceutical products, and at least one antenna operatively connected to a reader configured to interrogate the remotely readable tags, wherein the reader is operatively connected to a computerized inventory system that is configured to receive information about each remotely readable tag, and wherein the reader detects a position of each remotely readable tag and associated pharmaceutical product on the plurality of racks.

8. The wireless identification system of claim 7, wherein each of the plurality of pharmaceutical products are each stored in a separate secondary protective container.

9. The wireless identification system of claim 8, wherein the plurality of secondary protective containers comprise a plurality of apertures.

10. The wireless identification system of claim 7, wherein the storage device includes one or more doors sealed by door gaskets, and the connection between the antenna and the reader comprises a flat cable that is positioned above, between, or under the door gaskets.

11. The wireless identification system of claim 7, wherein the remotely readable tags comprise of RFID or MEMS.

12. A system for wireless identification of pharmaceutical products stored in a storage device comprising:
a plurality of racks adapted to hold a plurality of pharmaceutical products;
a plurality of remotely readable tags each adapted to be affixed to one or more of the plurality of pharmaceutical products,
a plurality of secondary protective containers that contain the plurality of pharmaceutical products, wherein the secondary protective containers comprise a plurality of apertures, and
at least one antenna operatively connected to a reader configured to interrogate the remotely readable tags,
wherein the reader is operatively connected to a computerized inventory system that is configured to receive information about each remotely readable tag, and wherein the reader detects a position of each remotely readable tag and associated pharmaceutical product on the plurality of racks.

13. The wireless identification system of claim 12, wherein the storage device includes one or more doors sealed by door gaskets, and the connection between the antenna and the reader comprises a flat cable that is positioned above, between, or under the door gaskets.

14. The wireless identification system of claim 12, wherein the remotely readable tags comprise of RFID or MEMS.

15. A system for wireless identification of pharmaceutical products stored in a storage device comprising:
a plurality of remotely readable tags affixed to a plurality of pharmaceutical products that are attached to a plurality of racks wherein the racks comprise a plurality of apertures, and
a multi-array antenna operatively connected to a reader configured to interrogate the remotely readable tags,
wherein the reader is operatively connected to a computerized inventory system that is configured to detect each remotely readable tag and wherein the reader detects a position of each remotely readable tag and associated pharmaceutical product on the plurality of racks.

16. The wireless identification system of claim 15, wherein the plurality of apertures on the plurality of racks are in alignment with the multi-array antenna.

17. The wireless identification system of claim 15, wherein each of the plurality of pharmaceutical products are stored in a separate secondary protective container.

18. The wireless identification system of claim 17, wherein the plurality of secondary protective containers comprise a plurality of apertures.

19. The wireless identification system of claim 15, wherein the storage device includes one or more doors sealed by door gaskets, and the connection between the antenna and the reader comprises a flat cable that is positioned above, between or under the door gaskets.

20. The wireless identification system of claim 15, wherein the remotely readable tags comprise of RFID or MEMS.

21. A system for a wireless identification of a pharmaceutical products stored in a storage device comprising:
a plurality of remotely readable tags associated with a plurality of pharmaceutical products on a plurality of racks wherein each rack functions as an antenna and is operatively connected to a reader configured to interrogate the remotely readable tags,
wherein the reader is operatively connected to a computerized inventory system that is configured to detect each remotely readable tag, and wherein the reader detects a position of each remotely readable tag and associated pharmaceutical product on each rack.

22. The wireless identification system of claim 21, wherein each of the plurality of pharmaceutical products are stored separately in a secondary protective container.

23. The wireless identification system of claim 22, wherein the secondary protective containers comprise a plurality of apertures.

24. The wireless identification system of claim 21, wherein the storage device includes one or more doors sealed by door gaskets, and the connection between the antenna and the reader comprises a flat cable that is positioned above, between or under the door gaskets.

25. The wireless identification system of claim 21, wherein the remotely readable tags comprise of RFID or MEMS.
